(19) United States
(12) Patent Application Publication
(10) Pub. No.: US 2004/0254460 A1
(43) Pub. Date: Dec. 16, 2004

(54) Method and apparatus for ultrasound examination

(76) Inventors: Michael Richard Burcher, Birmingham (GB); Julia Alison Noble, Oxford (GB)

Correspondence Address: KALOW & SPRINGUT LLP, 488 MADISON AVENUE, 19TH FLOOR, NEW YORK, NY, 10022, US

(21) Appl. No.: 10/489,481

(22) PCT Filed: Sep. 10, 2002
(86) PCT No.: PCT/GB02/04115

(30) Foreign Application Priority Data
Sep. 11, 2001 (GB) 0121984.9

(51) Int. Cl. A61B 8/00

(52) U.S. Cl. 600/437

(57) ABSTRACT

An ultrasound apparatus and method of ultrasound examination in which the contact force between the ultrasound probe and the subject is measured and recorded. Because contact between the ultrasound probe and the subject deforms the underlying tissue, recordal of the contact force allows the deformation to be calculated. Then an inverse deformation can be calculated and used to correct the received signals to generate the signals which would have been obtained if there had been no contact between the ultrasound probe and the subject. The deformation of the subject may be predicted using a model, such as a finite element model.

### The present invention relates to a method and apparatus for ultrasound examination, such as imaging, and in particular to a method of enhancing the quality of results obtained using ultrasound.

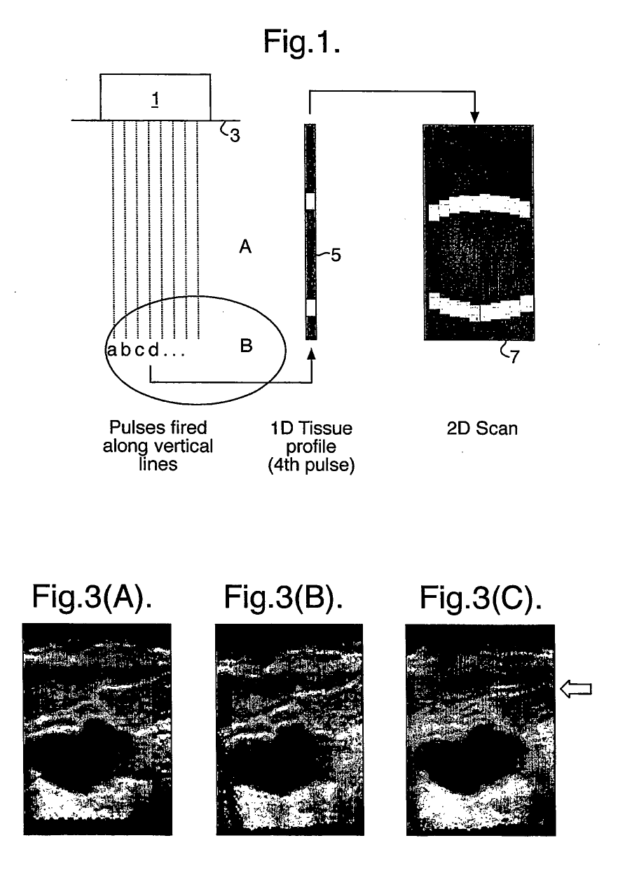

Ultrasound is regularly used to image soft tissues, such as tissues of the human or animal body in medical imaging, non-invasive inspection of industrial parts such as aircraft engine components, and some types of food in the field of food quality control and analysis. FIG. 1 of the accompanying drawings illustrates schematically how a conventional ultrasound image is created. An ultrasound probe 1 is placed in contact with the surface 3 of the subject and a plurality of ultrasound pulses a, b, c, d . . . etc are transmitted into the subject. The internal structure A, B of the subject gives it a varying acoustic echogenicity with depth, and so each pulse results in echoes received back at the ultrasound probe 1, these echoes forming one-dimensional profiles in which brightness is correlated with the acoustic echogenicity. A plurality of the one-dimensional profiles 5 can be assembled side by side to create a two-dimensional scan or slice 7.

Because the ultrasound probe must be placed in contact via gel or gel pads with the surface of the subject being imaged (in order to acoustically couple the probe to the subject), where the subject comprises soft tissue, the surface of the subject and the soft tissues beneath are deformed. In some fields, such as conventional diagnostic two-dimensional ultrasound scanning, for instance for breast cancer diagnosis, the probe can be used to palpate a lesion while observing the resulting changes in the ultrasound image. This gives qualitative diagnostic information about the elastic properties of the tissues and the mobility of the lesion. Additional diagnostic information is contained in the appearance of the lesion on the ultrasound image, such as the roughness of its border (or margin) or its brightness (echogeneity). In general, these characteristics will not be affected by the distortion.

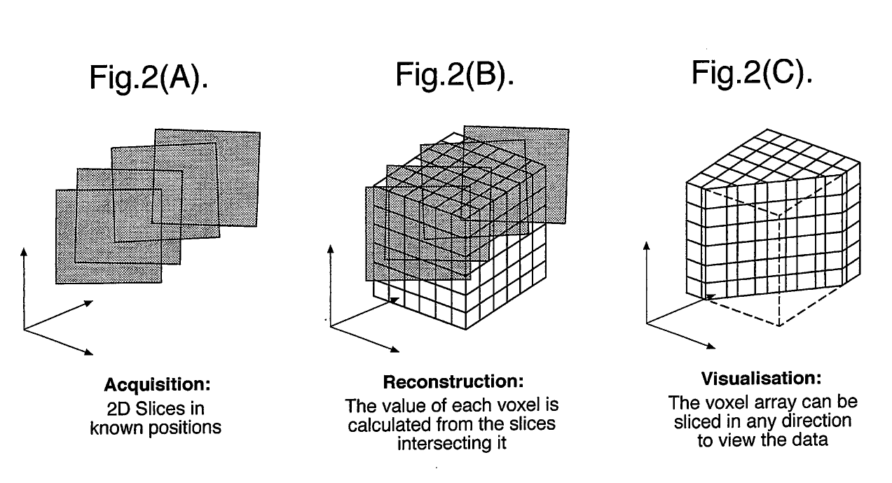

It is possible to use ultrasound signals from a subject to form a three-dimensional representation of the internal structure of that subject. This process is illustrated in FIG. 2 of the accompanying drawings. In this technique, so-called 3-D ultrasound imaging, a plurality of two-dimensional slices in known positions are acquired, as illustrated schematically in FIG. 2(a). Then, for each voxel (volume element) in the volume being imaged, an average value of the acoustic echogenicity can be obtained from all the slices intersecting it. These may be assembled together as illustrated in FIG. 2(b) to form the 3-D representation of the internal structure. This structure may be visualised by reslicing the three-dimensional array in any direction as illustrated in FIG. 2(c) and viewing the voxels that intersect the slice. It will be appreciated, though, that the reconstruction is accurate only if the shape of the tissue is the same in each of the two-dimensional scans. This assumption of constant shape is made in conventional imaging. By imaging the same tissue from more than one direction and combining the component sweep scans, a process known as “compounding”, the effects of image noise and potentially other artifacts can be reduced. However if the tissue has been distorted differently in the individual sweep scans, then compounding tends to give blurred images in which the borders of structures, such as lesions, are less clear than in the individual sweep scans, and curvilinear structures within the images are not aligned. This can be seen in FIG. 3 of the accompanying drawings in which two separate two-dimensional images are shown in FIGS. 3(a) and 3(b) (each is a slice through a 3D reconstruction from one sweep scan), and the compounded data set is illustrated in FIG. 3(c). It can be seen that the borders are less clear and structures within the images such as the bright near-horizontal line above the lesion (arrowed) are not aligned.

One way of ensuring that there is no change in shape during a scan is to apply the same force and distortion to the tissue during the scan. This can be achieved by using a mechanical sweep probe, or by constraining the subject and scanning through a window. However the geometry of the scan is restricted in both of these cases, and this limits their utility. Also, the tissue is still subject to an unknown deformation, which makes registration of ultrasound images with images from different modalities (such as x-rays, CT, MRI, PET, SPECT, etc), or across longitudinal data sets, difficult.

Image-based (normally meaning intensity-based) registration techniques can be used to align structures in the component images and reduce blurring, but one of the component scans must be used as a reference and so these methods are not capable of recovering the undistorted shape of the tissue.

The present invention provides a method and apparatus which allows reconstruction of the undeformed shape of tissue being imaged, that is to say which allows the production of the image which would have been achieved if there had been no contact between the subject and ultrasound probe.

In more detail the present invention provides a method of ultrasound examination comprising applying ultrasound to a subject and receiving ultrasound signals from the subject, by use of an ultrasound transducer, measuring the contact force between the transducer and the subject, further comprising the step of correcting the received signals for deformation of the subject caused by said contact.

The invention extends to a method of correcting pre-existing data sets, in other words a method which does not involve the step of applying the ultrasound to the subject.

Another aspect of the invention provides an apparatus for ultrasound examination of a subject, comprising an ultrasound transducer for contact with a subject being imaged and a force transducer for measuring the contact force between the transducer and subject, and a data processor responsive to the force transducer to correct ultrasound signals received from the subject for deformations of the subject caused by said contact.

In this context the term “contact” includes contact via an acoustic coupling medium such as gel or a gel pad.

The correction may be based on the contact force between the transducer and the subject, and optionally also the position of the transducer, which may be measured by using stereo cameras to measure the position of four LED's (light emitting diodes) mounted on the transducer. Other ways of measuring the position are, of course, possible.

The correction may comprise calculating the displacement of the surface of the subject as a function of the contact force, and preferably position, and then displacing the received signals spatially by an amount equal to the surface displacement. Alternatively, or in addition, the deformation of the internal structure of a subject may be calculated as a function of the contact force, and preferably position, and the correction applied may represent a reverse (also called an inverse) deformation of that applied.

The deformation of the internal structure may be calculated either from a finite element model of the subject, or by examining how the structure of a physical model or representation of the subject deforms under known forces, or by examining a plurality of ultrasound data of the subject obtained with different contact forces.

Other methods of deformation prediction, such as non-FEM-based analytical solutions, approximations (such as stretching the image), finite-difference solutions, exemplar models, etc may be used.

A plurality of the corrected signals taken from different positions may be assembled together to form a three-dimensional representation of the internal structure of the subject. Because the signals have been corrected for the deformation caused by contact of the transducer with the subject, the three-dimensional reconstruction is more accurate and more clear. Further, the true position of the internal structure is represented, thus facilitating comparison with other imaging modalities.

The transducer may comprise an ultrasound probe having both a transmitter and receiver, or the transmitter and receiver may be separate, and separately in contact with the subject. In that case the contact force between one or both of the transmitter and receiver may be measured, e.g. by separate force transducers, and the received signals corrected accordingly.

The force transducer may measure the torque on the transducer, created e.g. by angling the transducer into the subject or in translation of the transducer across the subject, and the received signals may be corrected for deformation resulting from that torque.

The invention is applicable in the medical and industrial fields mentioned above.

The present invention will be further described by way of example with reference to the accompanying drawings in which:

FIG. 1 schematically illustrates the formation of a two-dimensional ultrasound image;

FIG. 2 schematically illustrates the reconstruction and visualisation of a three-dimensional ultrasound image;

FIGS. 3(a) and (b) show two-dimensional ultrasound images and FIG. 3(c) shows the result of compounding the two images;

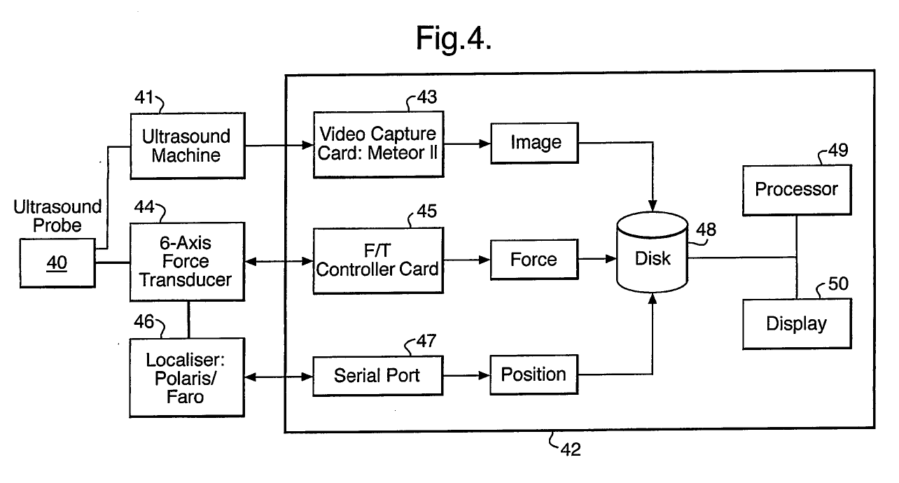

FIG. 4 schematically illustrates the system architecture of an embodiment of the invention;

FIG. 5 schematically illustrates an embodiment of an ultrasound probe used in the present invention;

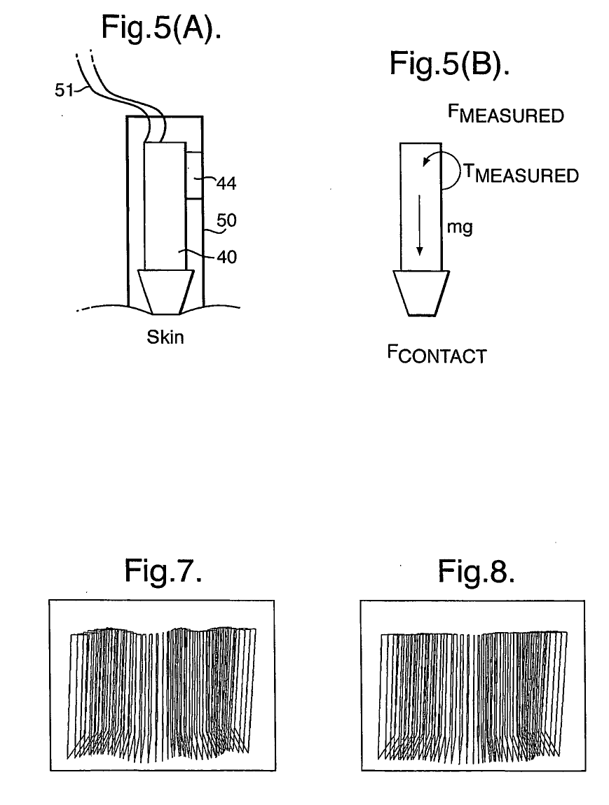

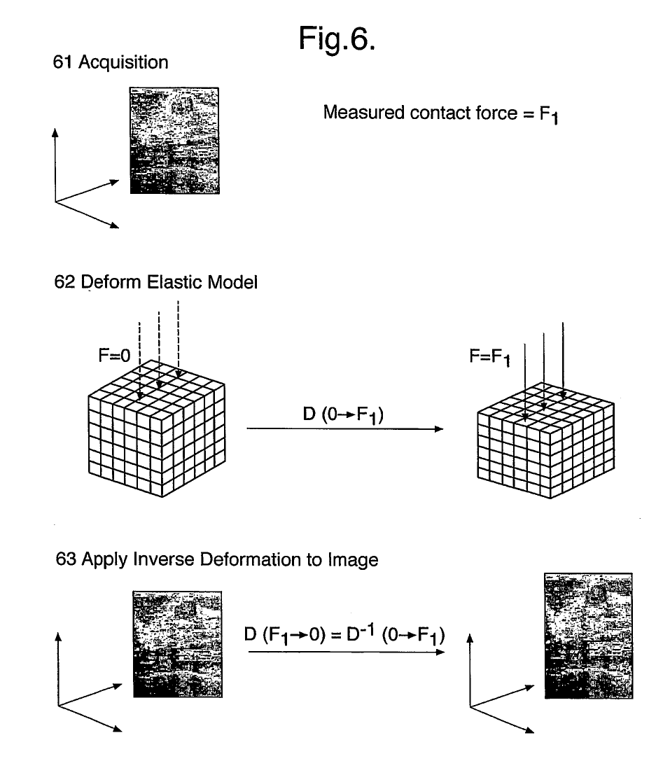

FIG. 6 schematically illustrates the process of deformation correction according to one embodiment of the invention;

FIG. 7 illustrates an assembly of two-dimensional images before correction;

FIG. 8 illustrates an assembly of two-dimensional images after correction;

FIGS. 9(a) to (c) illustrate ultrasound images of a phantom model to which the invention has been applied;

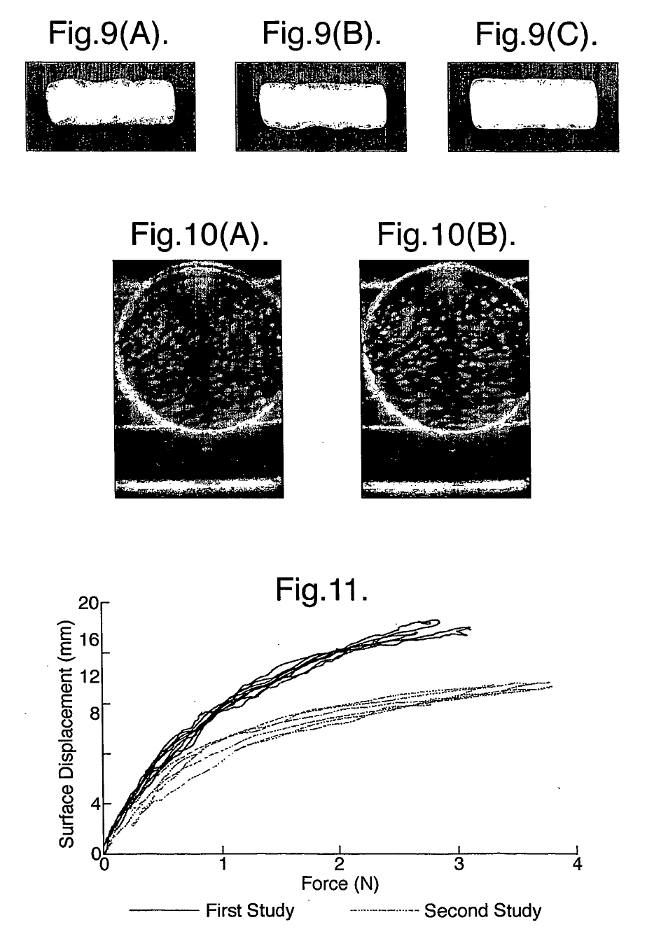

FIGS. 10(a) and (b) show reconstructions of the phantom model; and

FIG. 11 shows the relationship between contact force and surface displacement in the breast of a human volunteer for two studies with 14 weeks separation.

Ultrasound imaging apparatus in accordance with one embodiment of the invention is schematically illustrated in FIG. 4. It comprises an ultrasound probe 40, such as a 7.5 megahertz linear array probe (HP L7540, Agilent Technologies) and an ultrasound machine 41 such as an Agilent Technologies Sonos 5500. In this embodiment the images are processed and displayed by a conventional personal computer 42 which is provided with a frame grabber 43 (such as the Meteor II-MC frame grabber by Matrox Imaging, Dorval, Canada) which grabs frames from the video output of the ultrasound machine 41. As will be explained below, a force transducer 44 and position sensor 46 are provided for monitoring the contact force between the probe and subject and the position of the probe respectively. The force and position signal are input to the personal computer 42 via a force transducer controller card 45 and serial port 47 and the image, force and position signals are then stored on the data storage medium 48 of the computer. The computer also comprises a processor 49 and display 50 for processing and displaying the image signals. In this embodiment the position and force signals are obtained at sampling rates of 60 Hertz and the video at 25 Hertz. The signals may be acquired asynchronously, and the position and force measurements then interpolated to find the position and force at the time of image acquisition.

An example of the position sensor 46 is the Polaris hybrid optical tracker (Northern Digital Inc., Ontario, Canada) which uses stereo cameras to measure the position of four infrared led's mounted on the probe. The contact force may be measured using a 6-axis force transducer (Mini 40, ATI Industrial Automation, North Carolina, USA) which measures force with a resolution of 1.25 mN and to an absolute accuracy of ±0.2 N though a simple force transducer such as a load cell may be used or a distributed force sensor mounted directly on the ultrasound transducer head or an array of force transducers in the ultrasound transducer head itself. One embodiment of the arrangement of the force transducer 44 and ultrasound probe 40 is shown diagrammatically in FIG. 5(a). It can be seen that the ultrasound probe is positioned inside an enclosing box 50 to which it is attached by the force transducer 44. The cable 51 for the ultrasound probe is clamped to the box so that any forces applied to the cable will not be recorded by the force transducer 44. FIG. 5(b) shows a free body diagram of the probe. Because the probe is moved slowly during an image acquisition, its acceleration can be ignored and so for equilibrium vector sum of the probe weight, the measured force and contact force between the probe and the subject is zero. The contact force is then calculated by negating the sum of the measured force and probe weight. The transducer 44 also measures any torque on the probe. Normally there would be little or no torque, but sometimes a torque is deliberately applied by angling the probe into the tissue of the subject. Torque can also change in a characteristic way (such as an increase first in one direction and then in the other) as a probe is passed over tissue containing a harder area, such as a lesion.

By using this apparatus the contact force (which here is intended to mean force and any torque) between the probe and subject is known for each position of the transducer for each acquired image. This knowledge allows the images to be corrected for the deformation of the soft tissue of the subject as will be explained below.

In accordance with one embodiment of this invention the measured and recorded contact forces for an image sequence are applied to a mathematical elastic model which represents the mechanical behaviour of the tissue being imaged. Thus as illustrated in step 62 of FIG. 6, the application of a measured contact force F₁ to an elastic model causes a deformation D which is an estimate of the deformation of the actual subject at the time of acquisition. When the force is removed from the model, it relaxes to its original undeformed state and undergoes the inverse deformation D⁻¹. This inverse deformation D⁻¹ can be applied to the image, as represented schematically in step 63, and changes both the position and content of the image. The resulting image is the scan that would have been obtained if there had been no contact force between the probe and subject. The individual image slices which have been subject to the inverse deformation can then be used in a conventional three-dimensional reconstruction.

It will be appreciated that the accuracy of the reconstruction depends on how well the elastic model represents the deformation of the actual subject.

A simple model can be used which models only the deformation of the surface of the subject and not the deformation of the underlying tissue. The relationship between the surface displacement and the contact force can be determined in a preliminary scan in which the probe is pressed against the surface, varying the force over the range that will be used during the acquisition and at a range of different positions. An example of the relationship between surface displacement and force for a human breast measured in two studies on the same volunteer at 14 weeks separation is shown in FIG. 11. The image acquisition scans are then performed, and a force measurement is recorded for each two-dimensional image slice. The surface displacement at the time of each image acquisition can be calculated from the measured contact force, for instance using FIG. 11, a model fitted to the curve, or a look-up table corresponding to it, and then in the three-dimensional reconstruction each two-dimensional image slice can be displaced by the surface displacement and correctly positioned. The top of the image will now lie on an undeformed surface. An example of this reconstruction is shown in FIGS. 7 and 8. FIG. 7 shows the profile of the original, uncorrected, scan in which each rectangle represents the outline of the ultrasound image in space. The top surface is wavy because of different forces applied during the different image acquisitions. FIG. 8 shows the profile after force correction using the surface displacement model. The top surface is now flat and corresponds to the undeformed shape of the object.

It is possible, however, to improve on this model. A problem with modelling the surface displacement alone is that it does not allow for deformation of the internal structure of the subject. FIG. 9 illustrates the results of applying the surface displacement model to ultrasound images of a cylindrical phantom made from gelatine with a cylindrical inclusion of a different gelatine mixture. FIG. 9(a) shows the uncorrected image, FIG. 9(b) the results of correcting using the surface displacement. Although the top surface is now flat, because the model does not account for internal deformation, the bottom surface is wavy.

One way of achieving a representation of the mechanical behaviour of the internal tissues of the subject is to use a finite element model (FEM) as the elastic model. A finite element model is created of the subject (for instance finite element models have been proposed and published for the human breast) and the surface of the finite element model is displaced by an amount equal to the surface displacement of the subject during imaging. (That surface displacement can be obtained from the force/surface displacement relationship such as FIG. 11). Where torques are involved these may be included as an angle of deformation. The deformation of the model can be observed and the inverse deformation calculated. This inverse deformation is then applied to the image to correct it.

FIG. 9(c) illustrates the result of corrected images using a finite element model of the gelatine phantom. It can be seen that both the top and bottom surfaces of the phantom are now represented as flat.

In calculating the corrections for the images one option is to solve the finite element model for each image slice using the exact probe position and all of the measured force components. If this is computationally too expensive, though, the displacement field for each slice can be interpolated from FEM solutions at a few lateral offsets (eg 4 lateral offsets) and fewer forces (eg 20 forces). This can be done assuming that each slice is roughly perpendicular to the sweep axis and that the deformation field can be predicted from the component of force parallel to the ultrasound beam.

FIGS. 10(a) and 10(b) show sections from reconstructions using either no force correction (FIG. 10(a)) or finite element correction (FIG. 10(b)). In each case the volume is reconstructed by compounding three sweep scans taken with different constant forces. The average of all pixels intersecting a given voxel is used to set the voxel value. The reconstruction without force correction shown in FIG. 10(a) has misregistration artifacts, especially at the top of the image. Three separate outlines of the gelatine cylinder's upper surface, one from each of the component sweep scans. In FIG. 10(b) the images have been corrected using a finite element model to predict the deformation. The edges are now brought into alignment and the misregistration artifacts are reduced, giving a clearer compounded image.

Other models may also be used. One example is to use an empirical deformation model which is similar in concept to the finite element model, but models the observed deformation rather than the (theoretical or observed) material properties. This method can model structures and motions too complicated to be dealt with by finite element techniques (such as complicated non-linear, anisotropic materials and changing topologies). The deformation behaviour of the object could be observed using ultrasound or other non-invasive (non-destructive) imaging modalities (including optical, MRI, x-ray) or destructive testing methods. The measured contact force and probe location are used to index the appropriate deformation. The inverse deformation is then used to reconstruct the undeformed object as with the finite element model.

In the embodiment above the correction has been applied to the image. The image is formed by a representation of the amplitude of the returning ultrasound signals (which are at radio frequency). The correction may be applied to the r.f. signals directly, before formation of the image. In some applications the r.f. signals are analysed and used (for instance because they include phase information which can be useful). Thus by modelling how the r.f. data changes with the applied contact force (this is the idea underlying elastography) the measured contact force is then used to correct the r.f. data. Alternatively, the r.f. data can be combined with the contact force to derive additional information about the object being imaged. For example, the model of how r.f. data changes with force may include a number of parameters relating to the imaged object (density, scatterer size, temperature etc). These properties can be measured by finding the values that best fit the observed r.f. data and force measurements.

The invention may be applied in the field of Doppler ultrasound. Moving objects within an ultrasound image can be detected by measuring the Doppler shift of their echoes. If the contact force on the probe changes, then it may move relative to the object, causing an artefactual Doppler signal. If the contact force is measured, then the motion this causes within the imaged tissue can be predicted using a model of tissue deformation and dynamics. This artefact can then be subtracted from the measured Doppler signal to obtain the true motion.

The acquisition of force data with the images gives a number of other advantages in addition to improving the compounding of the images. For instance, by recording the force and deformation of the tissue it is possible to obtain absolute values of the Young's Modulus of the tissue without knowing (or assuming) the density of the material. This can be useful in diagnosis. Thus the invention may be used in an improved method of elastography allowing simultaneous recording of force and displacement information. Further, the recordal of force can be used in training operators of ultrasound probes to sweep the probes with a constant force, thus improving the likelihood of accurate compounding of the images.

The presence of the force transducer can also be used to detect, and correct, the ultrasound signal for other physical processes which affect its quality e.g. heartbeat, breathing, vibrations etc.

1. Apparatus for ultrasound examination of a subject, comprising an ultrasound transducer for contact with a subject being imaged and a force transducer for measuring the contact force between the transducer and subject, and a data processor responsive to the force transducer to correct ultrasound signals received from the subject for deformations of the subject caused by said contact.

2. Apparatus according to claim 1 further comprising a position sensor for sensing the position of the transducer, and wherein the data processor is further responsive to the position sensor in correcting the signals.

3. Apparatus according to claim 1 wherein the correction comprises calculating the displacement of the surface of the subject as a function of the contact force and displacing the signals spatially by an amount equal to said displacement.

4. Apparatus according to claim 1, wherein the correction comprises calculating the deformation of the internal structure of the subject as a function of the contact force and applying a correction representing the reverse deformation.

5. Apparatus according to claim 4 wherein the deformation of the internal structure of the subject as a function of the contact force is calculated from a model of the subject.

6. Apparatus according to claim 4 wherein the deformation of the internal structure of the subject as a function of the contact force is calculated from a physical representation of the subject.

7. Apparatus according to claim 4 wherein the deformation of the internal structure of the subject as a function of the contact force is calculated from a plurality of signals obtained with different contact forces.

8. Apparatus according to claim 1 wherein the data processor is adapted to assemble from a plurality of the corrected signals taken from different positions a three-dimensional representation of the internal structure of the subject.

9. Apparatus according to claim 1 wherein the transducer comprises a transmitter and receiver housed within an ultrasound probe.

10. Apparatus according to claim 1 wherein the transducer comprises a separate transmitter and receiver, separately in contact with the subject and the force transducer measures the contact force of at least one of said transmitter and receiver.

11. Apparatus according to claim 1 wherein the data processor is adapted to process the ultrasound signals to form ultrasound image signals and wherein said image signals are corrected for said deformation.

12. Apparatus according to claim 1 wherein the force transducer is for measuring torque exerted on the ultrasound transducer.

13. A method of ultrasound examination comprising applying ultrasound to a subject and receiving ultrasound signals from the subject, by use of an ultrasound transducer, measuring the contact force between the transducer and the subject, further comprising the step of correcting the received signals for deformation of the subject caused by said contact.

14. A method of processing ultrasound signals from a subject obtained by use of an ultrasound transducer in contact with the subject, comprising the steps of measuring the contact force between the transducer and the subject and correcting the received signals for deformation of the subject caused by said contact between the transducer and the subject.

15. A method according to claim 14 wherein the step of correcting the received signals comprises applying a correction based on the contact force between the transducer and the subject.

16. A method according to claim 14 wherein the step of correcting the received signals comprises applying a correction based on the contact force between the probe and the subject and the position of the probe.

17. A method according to claim 14 wherein the correction comprises calculating the displacement of the surface of the subject as a function of the contact force and displacing the received signals spatially by an amount equal to said displacement.

18. A method according to claim 14 wherein the correction comprises calculating the deformation of the internal structure of the subject as a function of the contact force and applying a correction representing the reverse deformation.

19. A method according to claim 18 wherein the deformation of the internal structure of the subject as a function of the contact force is calculated from a finite element model of the subject.

20. A method according to claim 18 wherein the deformation of the internal structure of the subject as a function of the contact force is calculated from a physical representation of the subject.

21. A method according to claim 18 wherein the deformation of the internal structure of the subject as a function of the contact force is calculated from a plurality of signals received from the subject obtained with different contact forces between said transmitter and the subject.

22. A method according to claim 3 wherein the step of measuring the contact force comprises measuring torque exerted on the ultrasound transducer.

23. A method according to claim 13 further comprising assembling from a plurality of the corrected signals taken from different positions a three-dimensional representation of the internal structure of the subject.

24. A computer program comprising program code means for executing the method of claim 14.
